(19) United States
(12) Patent Application Publication
(10) Pub. No.: US 2018/0250359 A1
(43) Pub. Date: Sep. 6, 2018

(54) COMBINATION THERAPY USING GLIADIN AND GAMMA AMINOBUTYRIC ACID

(71) Applicant: DIAMYD MEDICAL AB, Stockholm (SE)

(72) Inventor: Anders ESSEN-MÖLLER, Stockholm (SE)

(21) Appl. No.: 15/753,935

(22) PCT Filed: Aug. 30, 2016
(86) PCT No.: PCT/SE2016/050809

§ 371 Date: Feb. 20, 2018

(30) Foreign Application Priority Data
Aug. 30, 2015 (SE) 1530128-6
Nov. 13, 2015 (SE) 1551469-8

(51) Int. Cl. A61K 38/16; A61K 9/00; A61P 1/00; A61P 3/10; A61P 25/16; A61P 25/28; A61P 37/02

(52) U.S. Cl. CPC A61K 38/168; A61K 9/0043; A61P 1/00; A61P 3/10; A61K 31/197; A61P 25/28; A61P 37/02; A61K 2300/00; A61P 25/16

(57) ABSTRACT

The present invention relates to the use of gliadin in medicine, in combination with gamma aminobutyric acid, and/or an antigen such as a beta cell autoantigen.

### FIELD OF THE INVENTION

The present invention relates to the field of medicinal products and their use in therapy of disease, in particular coeliac disease and diabetes. More specifically, the invention relates to the combination of gliadin with gamma aminobutyric acid and/or antigens including beta cell antigens in the treatment or prevention of certain disease.

### BACKGROUND

The use of gliadin or derivatives thereof for use in preventing or treating diabetes is disclosed in EP2187926.

The use of a combination of three specific gluten derived-peptides in the treatment of coeliac disease is disclosed in WO2010/060155.

Funda and co-workers (Funda, 2013) found that intranasal vaccination with gliadin may represent a novel, safer strategy for prevention or even early cure of T1D.

GABA-A receptors are the most abundant subtype of GABA receptor. GABA-A receptors are ionotropic receptors comprised of multiple subunits that form ligand-gated chloride ion channels. Activation of GABA-A receptors results in the passive diffusion of negative chloride ions into the cell, which increases the negative resting membrane potential (creating an inhibitory postsynaptic potential (IPSP)), rendering the cell more resistant to depolarization. In humans, seven classes of GABA-A receptor subunits have been cloned (alpha, beta, gamma, delta, epsilon, pi, and theta subunits), each encoded by a separate gene. In addition, many subunits have multiple isoforms and/or splice variants, giving rise to a large degree of structural diversity (see e.g., Simon et al., J Biol Chem., 279(40):41422-35 (2004)).

GABA-B receptors are widely distributed in the CNS, as well as the autonomic nerves of the PNS. GABA-B receptors are metabotropic, G-protein coupled receptors (GPCRs) of the seven-transmembrane family, and are functionally linked to potassium and/or calcium ion channels. Activation of presynaptic GABA-B receptors inhibits the influx of calcium, resulting in the inhibition of the release of GABA and/or other neurotransmitters by presynaptic neurons. Activation of postsynaptic GABA-B receptors opens potassium channels, resulting in an efflux of potassium out of the cell and an increase in the negative resting membrane potential. The GABA-B mediated response is a ‘slow’ response that underlies the late phase of the IPSP, whereas the GABA-A mediated response is a ‘fast’ response that underlies the early phase of the IPSP. GABA-B receptors can also modulate the activity of adenylyl cyclase, resulting in a variety of downstream responses. There are two GABA-B receptor subunits encoded by separate genes, termed GABA-B1 and GABA-B2 (sometimes referred to as GBR1 and GBR2, respectively), each of which gives rise to multiple splice variants. GABA-B receptors generally have a heterodimeric subunit composition (B1-B2).

GABA-C receptors are ionotropic receptors similar in structure and function to GABA-A receptors, but with a distinct subunit composition, distribution, and pharmacology. GABA-C receptors, like GABA-A receptors, are pentameric ligand-gated chloride ion channels. However, GABA-C receptors are comprised of a distinct subunit type, termed rho subunits, which exist in three isoforms. GABA-C receptors are primarily expressed in the retina, although the mRNA of certain rho subunits is more widely distributed throughout the CNS. Rho subunits have demonstrated the ability to form functional receptors in combination with GABA-A subunits in vitro, suggesting the possibility of additional combinations with unknown structure and function.

### SUMMARY

The invention is as set out in the appended claims.

### Definitions

All terms and expressions as used herein are intended to have the meaning given to them by the person skilled in the art at the filing date of the present application, unless any other expression is evident from the context of this disclosure. However, for the sake of clarity, some terms and expressions are explicitly defined below.

The term “gliadin” refers to the gliadin protein found in wheat, and incorporates gliadin derivatives such as alpha-gliadin, beta-gliadin, gamma-gliadin, omega-gliadin, deamidated gliadin, and tissue transglutaminase (tTG) treated gliadin and fragments thereof.

The term “glutamic acid decarboxylase”, abbreviated GAD, is a major beta cell auto-antigen. It appears in a 65 kDa form referred to as GAD65 and a 67 kDa form referred to as GAD67 and these forms are included in the term GAD.

The terms “gamma-amino butyric acid analogs” and “GABA agent” may be used interchangeably and refers generally, as used herein, to a compound that directly or indirectly modulates the activity of GABA receptor relative to the activity of the GABA receptor in the absence of the compound. “GABA agents” useful in the invention described herein include compounds or agents that, under certain conditions, may act as modulators of GABA receptor activity, such as vigabatrin and baclofen and any GABA A or GABA B receptor agonist. In some embodiments, the activity of a GABA modulator is assessed relative to an agent known to have a particular effect on GABA receptors under certain conditions (i.e., “prototypical” modulators). Examples of prototypical agonists for GABA-A, GABA-B, and GABA-C receptors are muscimol (which also acts as a GABA-C partial agonist), baclofen, and cis-aminocrotonic acid (CACA), respectively. Examples of prototypical antagonists for GABA-A, GABA-B, and GABA-C receptors are bicuculline, CGP 64213, and 1,2,5,6-tetrahydropyridine-4-yl methyl phosphinic acid (TPMPA), respectively. Additional prototypical GABA modulators are known in the art.

An “autoantigen” or “self-antigen” is an endogenous tissue constituent that has the ability to interact with autoantibodies and cause an immune response. A “beta cell autoantigen” is an autoantigen originating from pancreatic beta cells. Beta cell autoantigens include, but are not limited to, glutamic acid decarboxylase (GAD), insulinoma antigen-2, ZnT8, islet-specific glucose-6-phosphate catalytic subunit-related protein (IGRP), chromogranin A, insulin, B chain insulin, proinsulin, and preproinsulin.

An “autoantibody” is an antibody that reacts with autoantigens of the organism that produced them.

The term “Vitamin D” includes vitamin D₂ and vitamin D₃. “Vitamin D analogs” include without prejudice Ergocalciferol, Dihydrotachysterol, Alfacalcidol, Calcitriol, Colecalciferol, and Calcifediol, and combinations thereof, as well as any other vitamin D analog classified in group A11CC of the Anatomical Therapeutic Chemical Classification System.

The term “cyclooxygenase inhibitors”, or “cox inhibitor”, relates to compounds that combine with cyclooxygenase and thereby prevent its substrate-enzyme combination with arachidonic acid and the formation of eicosanoids, prostaglandins, and thromboxanes. A subgroup of the cyclooxygenase inhibitors is the cyclooxygenase-2 inhibitors, which have specificity for cyclooxygenase-2.

The term “TNF alpha inhibitor” relates to compounds that inhibits the action of Tumour Necrosis Factor alpha (TNF alpha), and includes adalimumab, certolizumab, etanercept, golimumab, infliximab, as well as any other compound classified in group L04AB of the Anatomical Therapeutic Chemical Classification System.

An “epitope” is the surface portion of an antigen capable of eliciting an immune response and of combining with the antibody produced to counter that response, or a Tcell receptor.

Expressions using the singular “a”, “an” and the like shall be construed as including the plural.

As used herein, “coadministration” refers to administering the compounds of the regimen of the present invention so that their dosing regimens overlap. They do not need to be administered at the same time.

The abbreviation “T1D” stands for “Type 1 Diabetes”.

### DETAILED DESCRIPTION

In certain embodiments in any of the disclosed methods and/or compositions the composition comprises gliadin and a GABA agent and/or the composition administered is gliadin and a GABA agent. In certain embodiments in any of the foregoing methods and/or compositions the GABA agent is a GABA_{A}-specific agonist and/or GABA_{A} preferential and/or a GABA_{B}-specific and/or a GABA_{B}-preferential agonist. In certain embodiments in any of the foregoing methods and/or compositions the GABA receptor agonist comprises a compound selected from the group consisting of thiopental, thiamylal, pentobarbital, secobarbital, hexobarbital, butobarbital, amobarbital, barbital, mephobarbital, phenobarbital, primidone, midazolam, triazolam, lometazepam, flutazolam, nitrazepam, fluritrazepam, nimetazepam, diazepam, medazepam, oxazolam, prazeam, tofisopam, rilmazafonoe, lorazepam, temazepam, oxazepam, fluidazepam, chlordizaepoxide, cloxazolam, flutoprazepam, alprazolam, estazolam, bromazepam, flurazepam, clorazepate potassium, haloxazolam, ethyl loflazepate, qazepam, clonazepam, mexazolam, etizolam, brotizolam, clotizaepam, propofol, fospropofol, zolpidem, zopiclone, and exzopiclone.

In certain embodiments in any of the disclosed methods and/or compositions the GABA receptor agonist comprises a compound is selected from the group consisting of muscimol, THIP/gaboxadol, Isoguvacine, Kojic amine, GABA, Homotaurine, Homohypotaurine, Trans-aminocyclopentane-3-carboxylic acid, Trans-amino-4-crotonic acid, .beta.-guanidinopropionic acid, homo-.beta.-proline, Isonipecotic acid, 3-((aminoiminomethyl)thio)-2-propenoic acid (ZAPA), Imidazoleacetic acid, and piperidine-4-sulfonic acid (P4S).

In certain embodiments the GABA receptor agonist is not a barbituate, and/or not a benzodiazepine, and/or not a thienodiazepine, and/or not a dialkylphenol. In certain embodiments the compound (active agent) is not GABA.

In certain embodiments methods involving the coadministration of GABA and/or GABA agonists in combination with a second active agent are contemplated. In certain embodiments a method of delaying the onset of type I diabetes, and/or slowing the progression of type I diabetes, and/or reducing the severity of type I diabetes, and/or reversing type I diabetes in a mammal in a mammal is provided.

The method typically involves coadministering to said mammal a first compound (or composition) comprising GABA, a GABA analogue/agonist, and/or a GABA prodrug and/or A GABA agonist prodrug; and a second compound (or composition) comprising a non-GABA therapeutic agent for the treatment of type I diabetes, and/or an anti-inflammatory compound, and/or a factor that stimulates a regulatory or immune response, and/or a GABA.sub.B receptor agonist, where the first compound and the second compound are administered in an amount sufficient to delay the onset of type I diabetes, and/or slow the progression of type I diabetes, and/or reduce the severity of type I diabetes, and/or reverse type I diabetes in said mammal.

In certain embodiments a method of delaying the onset of an immune response, and/or slowing the progression of an immune response, and/or reducing the severity of an immune response, and/or suppressing an immune response in a mammal is provided. The method typically involves coadministering to the mammal a first compound (or composition) comprising GABA, a GABA analogue, a GABA agonist, a GABA or GABA agonist prodrug, and/or a GABA potentiator; and a second compound (or composition) comprising a non-GABA therapeutic agent for the treatment of type I diabetes, and/or an anti-inflammatory compound, and/or a factor that stimulates a regulatory or immune response, and/or a GABA.sub.B receptor agonist, where the first compound and the second compound are administered in an amount sufficient to delay the onset of an immune response, and/or slow the progression of an immune response, and/or reduce the severity of an immune response, and/or suppress an immune response in the mammal.

In various embodiments a combination of compounds with gliadin for use in the various indications described above (and further below herein) is provided. Accordingly, in certain embodiments first compound comprising GABA, a GABA analogue, a GABA agonist, and/or a GABA or GABA agonist prodrug; and a second compound comprising a non-GABA therapeutic agent for the treatment of type I diabetes, and/or an anti-inflammatory compound, and/or a factor that stimulates a regulatory or immune response, and/or a GABA.sub.B receptor agonist for use in conjunction with each other to delay the onset of type I diabetes, and/or slow the progression of type I diabetes, and/or reduce the severity of type I diabetes, and/or reverse type I diabetes in a mammal; and/or to promote transplanted islet cell survival in a mammal; and/or to delay the onset of hyperglycemia, and/or to slow the progression of hyperglycemia, and/or to reduce the severity of hyperglycemia, and/or to reverse hyperglycemia in a mammal; and/or to promote transplanted islet cell survival in a mammal having type I diabetes or at risk for type I diabetes who is a recipient of transplanted islet cells; and/or to delay the onset of an immune response, and/or slow the progression of an immune response, and/or reduce the severity of an immune response, and/or suppress an immune response in a mammal; and/or to protect beta-cells in a mammal from oxidative stress induced apoptosis is provided.

The gliadin component according to the invention can also comprise at least one of a first peptide comprising the amino acid sequence LQPFPQPELPYPQPQ, ii) a second peptide comprising the amino acid sequence QPFPQPEQPFPWQP, and iii) a third peptide comprising the amino acid sequence PEQPIPEQPQPYPQQ (SEQ ID NO: 16), or biologically active fragments or variants thereof. The gliadin component can consist of one, two or three of these peptides, or comprise further gliadin peptides, fragments or variants.

Also provided is a kit comprising a first container containing Gliadin and GABA, and/or a GABA analogue, and/or a GABA agonist, and/or a GABA or GABA agonist prodrug; and a second container containing a non-GABA therapeutic agent for the treatment of type I diabetes, and/or an anti-inflammatory compound, and/or a factor that stimulates a regulatory or immune response, and/or a GABA.sub.B receptor agonist.

Additionally, “combination” pharmaceutical formulations are provided. In certain embodiments the formulation comprises Gliadin, GABA, a GABA analogue, a GABA agonist, and/or a GABA or GABA agonist prodrug; and a non-GABA therapeutic agent for the treatment of type I diabetes, and/or an anti-inflammatory compound, and/or a factor that stimulates a regulatory or immune response, and/or a GABA.sub.B receptor agonist.

In certain embodiments in the various “combination” methods, compounds, kits, or formulations described above (and below herein) the GABA analogue, or GABA agonist is a GABA_{A} receptor specific or preferential agonist. In certain embodiments in the various “combination” methods, compounds, kits, or formulations described above (and below herein) the GABA analogue, or GABA agonist is a GABA.sub.B receptor specific or preferential agonist. In certain embodiments in the various “combination” methods, compounds, kits, or formulations described above (and below herein) the GABA analogue, or GABA agonist, is one that does not substantially cross the blood-brain barrier. In certain embodiments in the various “combination” methods, compounds, kits, or formulations described above (and below herein) the first compound is GABA. In certain embodiments in the various “combination” methods, compounds, kits, or formulations described above (and below herein) the first compound is selected from the group consisting of GABA, muscimol, THIP/gaboxadol, Isoguvacine, Kojic amine, Homotaurine, Homohypotaurine, Trans-aminocyclopentane-3-carboxylic acid, Trans-amino-4-crotonic acid, .beta.-guanidinopropionic acid, homo-.beta.-proline, Isonipecotic acid, 3-((aminoiminomethyl)thio)-2-propenoic acid (ZAPA), Imidazoleacetic acid, and piperidine-4-sulfonic acid (P4S). In certain embodiments in the various “combination” methods, compounds, kits, or formulations described above (and below herein) the first compound is selected from the group consisting of thiopental, thiamylal, pentobarbital, secobarbital, hexobarbital, butobarbital, amobarbital, barbital, mephobarbital, phenobarbital, primidone, midazolam, triazolam, lometazepam, flutazolam, nitrazepam, fluritrazepam, nimetazepam, diazepam, medazepam, oxazolam, prazeam, tofisopam, rilmazafonoe, lorazepam, temazepam, oxazepam, fluidazepam, chlordizaepoxide, cloxazolam, flutoprazepam, alprazolam, estazolam, bromazepam, flurazepam, clorazepate potassium, haloxazolam, ethyl loflazepate, qazepam, clonazepam, mexazolam, etizolam, brotizolam, clotizaepam, propofol, and fospropofol, zolpidem, zopiclone, and exzopiclone. In certain embodiments in the various “combination” methods, compounds, kits, or formulations described above (and below herein) the first compound is not a barbituate, and/or not a benzodiazepine, and/or not a thienodiazepine, and/or not a dialkylphenol. In certain embodiments in the various “combination” methods, compounds, kits, or formulations described above (and below herein) the first compound is not GABA.

In certain embodiments in the various “combination” methods, compounds, kits, or formulations described above (and below herein) the second compound is a .beta.-cell antigen and/or a nucleic acid encoding a .beta.-cell antigen and/or an immune cell specific immunosuppressant. In certain embodiments in the various “combination” methods, compounds, kits, or formulations described above (and below herein) the second compound comprises one or more .beta.-cell antigens or a nucleic acid encoding one or more of said antigens, wherein said antigens comprise an antigen selected from the group consisting of GAD (GAD65), GAD67, hsp65 or an immunogenic fragment thereof, an insulin b-chain or immunogenic fragment thereof, an HSPp277 or immunogenic fragment thereof, an MHC molecule from an islet donor cell or an immunogenic fragment thereof, proinsulin or an immunogenic fragment thereof, preproinsulin or an immunogenic fragment thereof, islet-specific glucose 6 phosphatase catalytic subunit-related protein (IGRP) or an immunogenic fragment thereof, chromogranin A or an immunogenic fragment thereof, insulinoma antigen-2 or an immunogenic fragment thereof, and ZnT8 or an immunogenic fragment thereof. In various embodiments the antigen is combined with an adjuvant (e.g., alum). In certain embodiments in the various “combination” methods, compounds, kits, or formulations described above (and below herein) the second compound comprises a composition selected from the group consisting of an antigen that has a therapeutic effect in type I diabetes, an anti-CD3 antibody, exendin-4, and a pro-insulin therapeutic. In certain embodiments in the various “combination” methods, compounds, kits, or formulations described above (and below herein) the second compound comprises a non-activating anti-CD3 monoclonal antibody. In certain embodiments in the various “combination” methods, compounds, kits, or formulations described above (and below herein) the second compound comprises a hOKT3.gamma.1(Ala-Ala) monoclonal antibody or an anti-CD3 F(ab′).sub.2. In certain embodiments in the various “combination” methods, compounds, kits, or formulations described above (and below herein) the second compound comprises a composition selected from the group consisting of Alpha-1 Antitrypsin (AAT), Canakinumab, Diamyd, Exsulin, LCT, Lisofylline, Rituximab, Xoma 052, DiaPep277, Prochymal, Reparixin, Thymoglobulin, Ilaris (canakinumab), JANUVIA® AND PREVACID®, Alpha-antitrypsin, and Amevive (alefacept). In certain embodiments in the various “combination” methods, compounds, kits, or formulations described above (and below herein) the compound comprises a factor that stimulates a regulatory and/or immune response (e.g., an interleukin-2 or an analogue thereof, TGF.beta. or an analogue thereof, IL-10 or an analogue thereof, an IL-6 antagonist, an IL-23 antagonist, a CD25 antagonist, an anti-IL-6 antibody, anti-IL-23 antibody, and an anti-CD25 antibody). In certain embodiments in the various “combination” methods, compounds, kits, or formulations described above (and below herein) the second compound has anti-inflammatory activity and/or is a regulator of an immune response. In certain embodiments in the various “combination” methods, compounds, kits, or formulations described above (and below herein) the second compound comprises a compound selected from the group consisting of an anti-CD3 antibody, anti-TNF, anti-IFN, CTLA-4 fused to Ig, anti-thymocyte globulin, anti-CD3 antibody (muromonab or Otelixizumab), sirolimus, and mycophenolate. In certain embodiments in the various “combination” methods, compounds, kits, or formulations described above (and below herein) the second compound comprises a compound selected from the group consisting of Azathioprine, Mycophenolic acid, Leflunomide, Teriflunomide, methotrexate, FKBP/Cyclophilin/Calcineurin, Tacrolimus, Ciclosporin, Pimecrolimus, Abetimus, Gusperimus, Thalidomide, Lenalidomide, Sirolimus, Deforolimus, Everolimus, Temsirolimus, Zotarolimus, Biolimus A9, and Anakinra In certain embodiments in the various “combination” methods, compounds, kits, or formulations described above (and below herein) the second compound comprises a compound selected from the group consisting of anti-Complement component 5 (Eculizumab), anti-TNFs (Infliximab, Adalimumab, Certolizumab pegol, Afelimomab, Golimumab, Nerelimomab), anti-Interleukin 5 (Mepolizumab), anti-imunoglobulin E (Omalizumab), anti-Interferon (Faralimomab), anti-IL-6 (Elsilimomab), anti-IL-12 and anti-IL-23 (Lebrikizumab, Ustekinumab), anti-CD3 (Muromonab-CD3, Otelixizumab, Teplizumab, Visilizumab), anti-CD4 (Clenoliximab, Keliximab, Zanolimumab), anti-CD11a (Efalizumab), anti-CD18 (Erlizumab), anti-CD20 (Afutuzumab, Rituximab, Ocrelizumab, Pascolizumab), anti-CD23 (Lumiliximab), anti-CD40 (Teneliximab, Toralizumab), anti-CD62L/L-selectin (Aselizumab), anti-CD80 (Galiximab), anti-CD147/Basigin (Gavilimomab), anti-CD154 (Ruplizumab), anti-BLyS (Belimumab), CTLA-4 (Ipilimumab, Tremelimumab), CAT (Bertilimumab, Lerdelimumab, Metelimumab), anti-Integrin (Natalizumab), anti-Interleukin-6 receptor (Tocilizumab), anti-LFA-1 (Odulimomab), anti-IL-2 receptor/CD25 (Basiliximab, Daclizumab, Inolimomab), anti-T-lymphocyte (Zolimomab aritox), Atorolimumab, Cedelizumab, Dorlixizumab, Fontolizumab, Gantenerumab, Gomiliximab, Maslimomab, Morolimumab, Pexelizumab, Reslizumab, Rovelizumab, Siplizumab, Talizumab, Telimomab aritox, Vapaliximab, Vepalimomab, Anti-thymocyte globulin, Anti-lymphocyte globulin, CTLA-4 (Abatacept, Belatacept), TNF inhibitor (Etanercept, Pegsunercept), Aflibercept, Alefacept, and Rilonacept. In certain embodiments second compound has no agonistic activity at a GABA receptor (e.g., at a GABA.sub.A receptor and/or at a GABA.sub.B receptor, and/or at a GABA.sub.C receptor). In certain embodiments the second compound has agonistic activity at a GABA.sub.B receptor. In certain embodiments the second compound has agonistic activity at a GABA.sub.B receptor and is selected from the group consisting of baclofen, (3-amino-2(S)-hydroxypropyl)methylphosphinic acid (CGP 44532), 3-aminopropyl(methyl)phosphinic acid (SKF 97541), and 3-aminopropylphosphonic acid (3-APA), (3-amino-2-fluoropropyl)phosphinic acid; (2R)-(3-amino-2-fluoropropyl)phosphinic acid; (2S)-(3-amino-2-fluoropropyl)phosphinic acid; (3-amino-2-fluoro-1-methylpropyl)phosphinic acid; (3-amino-2-oxopropyl)phosphinic acid; (2S)-(3-amino-2-hydroxypropyl)phosphinic acid; (R)-(3-amino-2-hydroxypropyl)phosphinic acid; and (3-amino-1-fluoro-2-hydroxypropyl)phosphinic acid. Illustrative species of Formula III include (3-amino-2-fluoropropyl)sulphinic acid, (2S )-(3-amino-2-fluoropropyl)sulphinic acid, (2R)-(3-amino-2-fluoropropyl)sulphinic acid, (2S )-(3-amino-2-hydroxypropyl)sulphinic acid, and (2R)-(3-amino-2-hydroxypropyl)sulphinic acid and (3-amino-2-oxopropyl)sulphinic acid. In certain embodiments in the various “combination” methods, compounds, kits, or formulations described above (and below herein) the first compound is administered before said second compound; or first compound is administered after said second compound. In certain embodiments in the various “combination” methods, compounds, kits, or formulations described above (and below herein) the first compound is administered simultaneously with said second compound (e.g., the first compound and the second compound can be combined in a single formulation or simply administered essentially simultaneously). In certain embodiments in the various “combination” methods, compounds, kits, or formulations described above (and below herein) the combination of the first compound and the second compound is synergistic. In certain embodiments in the various “combination” methods, compounds, kits, or formulations described above (and below herein) the mammal is a human. In certain embodiments in the various “combination” methods, compounds, kits, or formulations described above (and below herein) the mammal (e.g., human) is diagnosed as having type I diabetes or determined to be at risk for type I diabetes. In certain embodiments in the various “combination” methods, compounds, kits, or formulations described above (and below herein) the mammal (e.g., human) is diagnosed as having hyperglycemia or being at risk for hyperglycemia. In certain embodiments in the various “combination” methods, compounds, kits, or formulations described above (and below herein) the mammal (e.g., human) contains transplanted islet cells. In certain embodiments in the various “combination” methods, compounds, kits, or formulations described above (and below herein) the mammal is not a mammal diagnosed as having or at risk for one or more conditions selected from the group consisting of a sleep disorder or insomnia, CNS disorder (e.g., muscle relaxation in spinal spasticity) a cardiovascular disorders, asthma, a gut motility disorder (e.g., irritable bowel syndrome), a subject being treated with a prokinetic and/or anti-tussive agents, a subject treated for emesis, a subject diagnosed as having or at risk for an autoimmune disease (e.g., rheumatoid arthritis), a subject diagnosed as having or at risk for a neurophysiological or neurophsychiatric disorder, a subject as having or at risk for a psychiatric disorder (e.g., anxiety and/or depression) a subject diagnosed as having or at risk for Huntington's disease and/or Parkinson's disease, and/or a subject having or at risk for MS.

The invention also relates to a method for prevention and/or treatment of a chronic inflammatory and or autoimmune disorder, comprising administering a composition, said composition comprising gliadin. In one embodiment, the invention relates to a method for prevention and/or treatment of an autoimmune disease, comprising administering a composition, said composition comprising gliadin, to a subject having a serum vitamin-D level above 50 nanomole/liter.

The subject to be treated may have a serum D-vitamin level between 50-150 nanomole/liter, such as 60-100 nanomole/liter, 75-100 nanomole/liter or 100-1500 nanomole/liter.

The method above, may further comprise a pretreatment of the subject to adjust the serum vitamin-D level. The pre-treatment of the subject may comprise administration of vitamin-D and/or vitamin-D analogs, and/or exposure to UVB-radiation, preferably for between 7 to 90 days before administration of the composition comprising at least one gliadin to said subject. In one embodiment, the method comprises administration of vitamin-D and/or vitamin-D analogs in an amount of 7000-70000 IU/week for 3-48 months.

The method above, may also comprise administering a cyclooxygenase inhibitor to the subject. In one embodiment, the cyclooxygenase inhibitor is selected from the group consisting of Ibuprofen, Dexibuprofen, Naproxen, Fenoprofen, Ketoprofen, Dexketoprofen, Flurbiprofen, Oxaprozin, Loxoprofen, Indomethacin, Tolmetin, Sulindac, Etodolac, Ketorolac, Diclofenac, Aceclofenac, Nabumetone, acetylsalicylic acid, Diflunisal (Dolobid), Salicylic acid, Salsalate (Disalcid), Piroxicam, Meloxicam, Tenoxicam, Droxicam, Lornoxicam, Isoxicam, Mefenamic acid, Meclofenamic acid, Flufenamic acid, Tolfenamic acid, Celecoxib, Rofecoxib, Valdecoxib, Parecoxib, Lumiracoxib, Etoricoxib, and Nimesulide.

The method above may also comprise administering a CTLA4 compound, such as abatacept, to the subject.

The method above may also comprise administering a TNF-alpha inhibitor to the subject. The TNF-alpha inhibitor is selected from the group consisting of Adalimumab, Certolizumab, Etanercept, Golimumab and Infliximab.

In a further aspect, the invention relates to a composition comprising a plurality of particles, each having immobilised on its surface at least one first and at least one second antigen, wherein the first antigen is gliadin, and the second antigen is either a tolerogen or a beta cell autoantigen, the composition further optionally comprising pharmaceutically acceptable adjuvants, excipients, solvents, and/or buffers.

In one embodiment, all beta cell autoantigens are selected from the group consisting of glutamic acid decarboxylase (GAD), insulinoma antigen-2, ZnT8, islet-specific glucose-6-phosphate catalytic subunit-related protein (IGRP), chromogranin A, insulin, B chain insulin, preproinsulin or proinsulin.

In one embodiment, at least one second antigen is a tolerogen. The tolerogen may be a native human protein, such as IL-10, IL35, Zebularin, Human Serum Albumin or hemoglobin, or gamma-amino butyric acid.

In one embodiment, the particle is an aluminium hydroxide (alum) particle, a liposome, a nanoparticle, a gold particle, or a biodegradable particle.

In certain embodiments, each particle has immobilised on its surface 2, 3, 4, 5, 6, 7, 8, 9, or 10 different antigens selected from the group consisting of tolerogens, IL10, 1L35, Zebularin, a GABA Agent, gliadin and beta cell autoantigens.

In one aspect, the invention relates to the composition according to the invention, for use as medicament. In one aspect, the composition is for use in a method for prevention and/or treatment of an autoimmune disease, such as type 1 diabetes or autoimmune diabetes or inflammatory gut disease.

In one aspect, the method for prevention and/or treatment of a chronic inflammatory and or autoimmune disorder, comprising administering a composition, said composition comprising gliadin, according to the invention comprises administering to the subject a composition according to the invention as described above.

In one aspect, the method comprises administering the composition containing the gliadin by intralymphatic injection, injection directly into a lymph node, subcutaneous injection, intramuscular injection, intraperitoneal injection, intravenous injection, intranasal, transmucosal or sublingual application; or orally, including administration as tablets, pellets, granules, capsules, lozenges, aqueous or oily solutions, suspensions, emulsions, sprays or as reconstituted dry powdered form with a liquid medium. In particular embodiments, the method comprises administering the composition containing the gliadin by intralymphatic injection or injection directly into a lymph node.

In one embodiment of the method the gliadin is administered in an amount of 1-1000 μg, per occasion.

In one embodiment the method comprises administering the composition comprising the gliadin at least 2 times, more preferred at least 4 times and most preferred at least 5 times, each administration being at least 1 (one) day apart.

In one embodiment the method comprises administering the gliadin in increased doses over a period of weeks, or months.

In one embodiment the method comprises the composition containing the gliadin is administered daily in an initial treatment period of at least 5 days, and optionally 1-2 months apart in at least one subsequent treatment period of at least 5 days.

In one embodiment the method the amount of gliadin is increased from 1-5 μg per administration at the beginning of the treatment period to about 40-1000 μg per administration in the final administrations.

Furthermore, the invention relates to a method for prevention and/or treatment of an autoimmune disease, comprising administering gliadin to a subject, in increasing doses over a period of weeks, months, or years.

In one embodiment the method a composition containing the gliadin is administered 1-4 weeks apart in an initial treatment period of 3 to 4 months, and optionally 2-3 months apart in a continued treatment period of 6-9 months.

In one embodiment the method the amount of gliadin is increased from 1-5 μg per administration at the beginning of the treatment period to about 40-100 μg per administration in the final administration.

The method above may further comprise administering a cyclooxygenase inhibitor to the subject. The cyclooxygenase inhibitor may be selected from the group consisting of Ibuprofen, Dexibuprofen, Naproxen, Fenoprofen, Ketoprofen, Dexketoprofen, Flurbiprofen, Oxaprozin, Loxoprofen, Indomethacin, Tolmetin, Sulindac, Etodolac, Ketorolac, Diclofenac, Aceclofenac, Nabumetone, acetylsalicylic acid, Diflunisal (Dolobid), Salicylic acid, Salsalate (Disalcid), Piroxicam, Meloxicam, Tenoxicam, Droxicam, Lornoxicam, Isoxicam, Mefenamic acid, Meclofenamic acid, Flufenamic acid, Tolfenamic acid, Celecoxib, Rofecoxib, Valdecoxib, Parecoxib, Lumiracoxib, Etoricoxib, and Nimesulide.

The method above may further comprise administering a CTLA4 compound, such as abatacept, to the subject.

The method above may further comprise administering a TNF-alpha inhibitor to the subject. The TNF-alpha inhibitor is selected from the group consisting of Adalimumab, Certolizumab, Etanercept, Golimumab and Infliximab.

The method above may further comprise administering vitamin-D, vitamin-D analogs, tyrosine kinase inhibitors, gamma-amino butyric acid or a gamma-amino butyric acid analog to the subject, and/or exposing the subject to UVB-radiation.

The method above may further comprise administration of vitamin-D and/or vitamin-D analogs and/or exposure to UVB-light, is performed for between 7 to 90 days before administration of the composition comprising at least one gliadin to said subject, such as administration of vitamin-D and/or vitamin-D analogs in an amount of 7000-70000 IU/week for 3-48 months.

The content of all prior publications referenced herein are incorporated by reference in the present application.

### REFERENCES

Funda, D. (2013). Prevention or Early Cure of Type 1 Diabetes by Intranasal Administration of Gliadin in NOD Mice. PLoS One, 9(4), s. e94530.

1. A method for treating or preventing a disease in subject, comprising administering gliadin in combination with gamma aminobutyric acid (GABA) and/or a GABA analog to the subject.

2. The method of claim 1, wherein the disease is an autoimmune or inflammatory disease.

3. The method of claim 1, wherein the disease is an autoimmune or inflammatory disease and the method further comprises administering to the subject at least one antigen relevant to the inflammatory or autoimmune disease to be treated or prevented, a fragment thereof, derivative thereof, or a nucleic acid coding therefor.

4. The method of claim 1, wherein the disease is type 1 diabetes; Metabolic Disease, Irritable Bowel Disease (IBD); Chron's Disease; Coeliac Disease; Gluten Intolerance; Lupus, Sjogrens Disease, Hashimoto's Disease, Grave's Disease, Rheumatoid Arthritis and/or neurodegenerative disease including Alzheimer's disease, Parkinson's disease, ALS or Multiple Sclerosis.

5. The method of claim 1, wherein the gliadin and GABA and/or GABA analog are administered to a patient in a single composition.

6. The method of claim 1, wherein the gliadin and GABA and/or GABA analog are administered to a patient in separate compositions.

7. The method of claim 1, wherein the gliadin is administered to a patient by mucosal application, such intranasal, buccal, sublingual, ocular, rectal, or vaginal application.

8. The method of claim 7, wherein the gliadin is administered as an aerosol intranasally or by inhalation to the patient.

9. The method of claim 1, wherein the gliadin is administered to a patient orally or by injection, such as subcutaneous injection, intramuscular injection, intraperitoneal injection, intravenous injection, intradermal injection, including administration as tablets, pellets, granules, capsules, lozenges, aqueous or oily solutions, suspensions, emulsions, sprays or as reconstituted dry powdered form with a liquid medium.

10. The method of claim 1, wherein the GABA and/or GABA analog is administered to the patient by subcutaneous injection, intramuscular injection, intraperitoneal injection, intravenous injection, intranasal, transmucosal or sublingual application; or orally, including administration as tablets, pellets, granules, capsules, lozenges, aqueous or oily solutions, suspensions, emulsions, sprays or as reconstituted dry powdered form with a liquid medium.

11. The method of claim 3, wherein the gliadin and the antigen, a fragment or derivative thereof, or a nucleic acid coding for the antigen, are administered to a patient in a single composition.

12. The method of claim 3, wherein gliadin and the antigen, a fragment or derivative thereof, or a nucleic acid coding for the antigen, are administered to a patient in separate compositions.

13. The method of claim 3, wherein the antigen, a fragment or derivative thereof, or a nucleic acid coding for the antigen, is administered to the patient by subcutaneous injection, intramuscular injection, intraperitoneal injection, intravenous injection, intranasal, transmucosal or sublingual application; or orally, including administration as tablets, pellets, granules, capsules, lozenges, aqueous or oily solutions, suspensions, emulsions, sprays or as reconstituted dry powdered form with a liquid medium.

14. The method of claim 3, wherein the at least one antigen comprises at least one beta cell autoantigen, such as glutamic acid decarboxylase (GAD), insulinoma antigen-2, ZnT8, islet-specific glucose-6-phosphate catalytic subunit-related protein (IGRP), chromogranin A, insulin, B chain insulin, proinsulin, and preproinsulin or any fragment or nucleic acid thereof.

15. The method of claim 1, wherein the method further comprises administering at least one compound selected from the group consisting of Vitamin D, Kappaproct, TLR9-antagonists, TLR4 antagonists, TLR2 antagonists, Zebularin, IL10 and IL35.

16. The method of claim 1, wherein said gliadin comprises at least one of a first peptide comprising the amino acid sequence LQPFPQPELPYPQPQ, ii) a second peptide comprising the amino acid sequence QPFPQPEQPFPWQP, and iii) a third peptide comprising the amino acid sequence PEQPIPEQPQPYPQQ (SEQ ID NO: 16), or biologically active fragments or variants thereof.

17. (canceled)

18. A pharmaceutical composition comprising gliadin and at least one of
GABA and/or a GABA analog; and
an antigen including a beta cell autoantigen relevant for the disease to be treated, a fragment or derivative of said antigen, or a nucleic acid coding for said antigen;
and optionally pharmaceutically acceptable adjuvants, excipients, solvents, and/or buffers.

19. The pharmaceutical composition according to claim 18, comprising gliadin and GABA and/or a GABA analog, and optionally pharmaceutically acceptable adjuvants, excipients, solvents, and/or buffers.

20. The pharmaceutical composition according to claim 18, wherein said gliadin comprises at least one of a first peptide comprising the amino acid sequence LQPFPQPELPYPQPQ, ii) a second peptide comprising the amino acid sequence QPFPQPEQPFPWQP, and iii) a third peptide comprising the amino acid sequence PEQPIPEQPQPYPQQ (SEQ ID NO: 16), or biologically active fragments or variants thereof.

21. The pharmaceutical composition according to claim 18, which is for intranasal application.

22. A container cartridge comprising the pharmaceutical composition according to claim 18.

23. An aerosol dispenser comprising a container cartridge according to claim 22.
